(19) United States
(12) Patent Application Publication
(10) Pub. No.: US 2018/0055485 A1
(43) Pub. Date: Mar. 1, 2018

(54) USER INTERFACE AND DISPLAY FOR AN ULTRASOUND SYSTEM

(71) Applicant: CARESTREAM HEALTH, INC., Rochester, NY (US)

(72) Inventors: Michael C. Lalena, Webster, NY (US); Craig F. Hofmann, Fairport, NY (US)

(21) Appl. No.: 15/681,489

(22) Filed: Aug. 21, 2017

Related U.S. Application Data
(60) Provisional application No. 62/378,277, filed on Aug. 23, 2016.
(60) Provisional application No. 62/378,283, filed on Aug. 23, 2016.
(60) Provisional application No. 62/378,260, filed on Aug. 23, 2016.

(51) Int. Cl. A61B 8/00; G06F 19/00

(52) U.S. Cl. CPC A61B 8/461; A61B 5/7207; G06F 19/3406

(57) ABSTRACT

An ultrasound apparatus has a beamformer, a control logic processor, and a transducer probe configured to direct and detect ultrasound signals. A control panel is in wireless signal communication with the control logic processor. A display monitor is in wireless signal communication with the control logic processor.

### CROSS REFERENCE TO RELATED APPLICATIONS

This application claims the benefit of U.S. Provisional application U.S. Ser. No. 62/378,277, provisionally filed on Aug. 23, 2016, entitled “USER INTERFACE AND DISPLAY FOR AN ULTRASOUND SYSTEM”, in the name of Michael C. Lalena, incorporated herein in its entirety.

This application claims the benefit of U.S. Provisional application U.S. Ser. No. 62/378,283, provisionally filed on Aug. 23, 2016, entitled “ULTRASOUND SYSTEM HAVING CLEAN FEATURE”, in the name of Michael C. Lalena, incorporated herein in its entirety.

This application claims the benefit of U.S. Provisional application U.S. Ser. No. 62/378,260, provisionally filed on Aug. 23, 2016, entitled “ULTRASOUND SYSTEM HAVING CUSTOMIZED LOGIN”, in the name of Michael C. Lalena, incorporated herein in its entirety.

### TECHNICAL FIELD

The disclosure relates generally to the field of ultrasound imaging apparatus and more particularly to ultrasound apparatus design suited to operator and patient ergonomics.

### BACKGROUND

Ultrasound imaging systems/methods are well-known medical devices, such as those described, for example, in U.S. Pat. No. 6,705,995 (Poland), U.S. Pat. No. 5,370,120 (Oppelt), and U.S. Pat. No. 8,285,357 (Gardner), all of which are incorporated herein in their entirety. Various applications for diagnostic ultrasound systems are given, for example, in the article entitled “Ultrasound Transducer Selection In Clinical Imaging Practice”, by Szabo and Lewin, Journal of Ultrasound Medicine, 2013; 32:573-582, incorporated herein by reference in its entirety.

Ultrasound sensing utilizes sound waves at frequencies higher than those perceptible to the human ear. Ultrasonic images known as sonograms are generated as a result of pulsed ultrasonic energy that has been directed into tissue using a probe. The probe obtains echoed sound energy from the internal tissue and provides signal content that represents the different sound reflectivity exhibited by different tissue types. This signal content is then used to form images that visualize features of the internal tissue. Medical ultrasound, also known as diagnostic sonography or ultrasonography, is used as a diagnostic imaging technique used to help visualize features and operation of tendons, muscles, joints, vessels and internal organs of a patient.

An ultrasound device has a control panel and an image display. The control panel includes a plurality of control features/selectors, for example buttons, sliders, track ball, and the like. Medical practitioners recognize that regular cleaning of medical devices such as the control panel surfaces helps to reduce, eliminate, and/or prevent the spread of disease. Medical practitioners have also recognized that it is difficult to keep the control panel clean. In addition to control fixtures, some control panels also include one or more speakers where the speaker grill should also be cleaned; cleaning these devices can be difficult, particularly if the speaker grill includes voids, holes, or crevices.

In order to make ultrasound apparatus easier to use and more flexibly adaptable for diagnostic imaging, manufacturers continue to make these devices more compact and to improve ergonomic aspects of equipment setup and use. In particular, there have been a number of proposed solutions for adapting the operator display and control panel to the diagnostic environment, such as providing equipment surfaces that can be readily cleaned and disinfected. Attempts to improve ergonomics and workflow have been directed to features that allow the operator to perform the imaging tasks with more relaxed posture and allow improved visibility of imaging results for the patient.

While existing systems may have achieved certain degrees of success in their particular applications, there is a need for further improvement in adapting ultrasound equipment design to be better suited to diagnostic requirements and overall usability.

### SUMMARY

An object of the present disclosure is to address the need for improved ergonomics and usability for ultrasound apparatus.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

According to one aspect of the disclosure, there is provided an ultrasound apparatus comprising: a beamformer; a control logic processor; a transducer probe configured to direct and detect ultrasound signals; a control panel that is in wireless signal communication with the control logic processor; and a display monitor that is in wireless signal communication with the control logic processor.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a sonographer using an exemplary ultrasound system.

FIG. 4 shows a displayed ultrasound image having a region of interest, shown in grayscale.

FIG. 5 shows a displayed ultrasound image having a region of interest, wherein a portion of the region of interest is highlighted in color.

FIG. 6 is a top view showing an ergonomic arrangement for an ultrasound system.

FIG. 7A shows an exemplary controller that may be suitable for an ultrasound system.

FIG. 7B shows an exemplary controller that may be suitable for an ultrasound system.

FIG. 8 shows an exemplary controller that may be suitable for an ultrasound system.

FIG. 9 shows an exemplary controller that may be suitable for an ultrasound system.

FIG. 10 shows an exemplary controller that may be suitable for an ultrasound system.

FIG. 11 is a perspective view that shows a removable control panel.

FIG. 12 is a plan view showing a configurable control panel.

FIGS. 13A, 13B, and 13C show an ultrasound system configuration for docked and wireless use, respectively.

FIG. 14 is a schematic diagram showing components for wireless recharging of control panel portions according to an embodiment of the present disclosure.

FIG. 15 is a schematic diagram that shows some basic components for a wireless charging system that uses inductive charging or a similar wireless charging mechanism.

FIG. 16 shows an ultrasound system having an etched glass control panel.

FIG. 17 shows speaker grills with a distributed mode loudspeaker (DML)/Flat Panel Speaker.

FIG. 18 shows an ultrasound system suitable for customized login.

### DETAILED DESCRIPTION OF THE EMBODIMENTS

The following is a detailed description of the preferred embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” is used to describe the patient that is undergoing ultrasound imaging. The terms “sonographer”, “technician”, “viewer”, “operator”, and “practitioner” are used to indicate the person who actively operates the sonography equipment.

The term “highlighting” for a displayed element or feature has its conventional meaning as is understood to those skilled in the information and image display arts. In general, highlighting uses some form of localized display enhancement to attract the attention of the viewer. Highlighting a portion of a display, such as a particular value, graph, message, or other element can be achieved in any of a number of ways, including, but not limited to, annotating, displaying a nearby or overlaying symbol, outlining or tracing, display in a different color or at a markedly different intensity or grayscale value than other image or information content, blinking or animation of a portion of a display, or display at larger scale, higher sharpness, or contrast.

### Overview of Ultrasound Apparatus and Technology

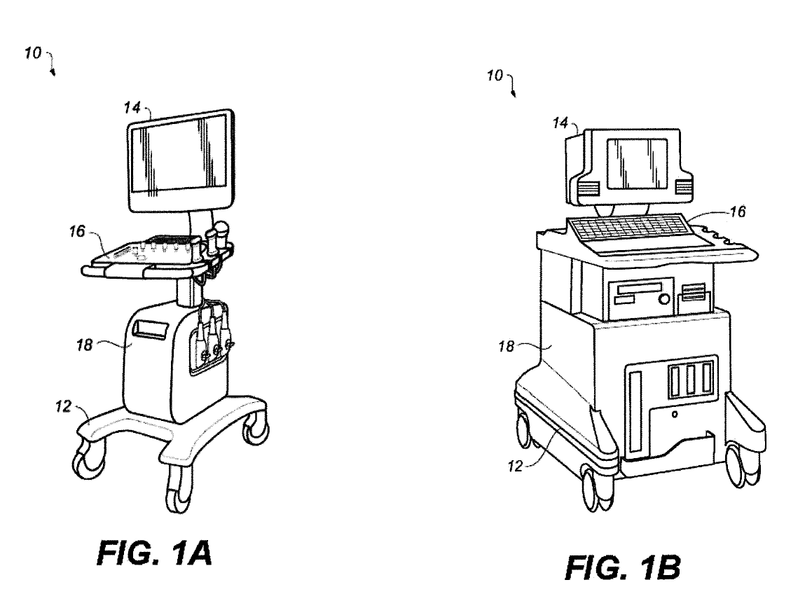

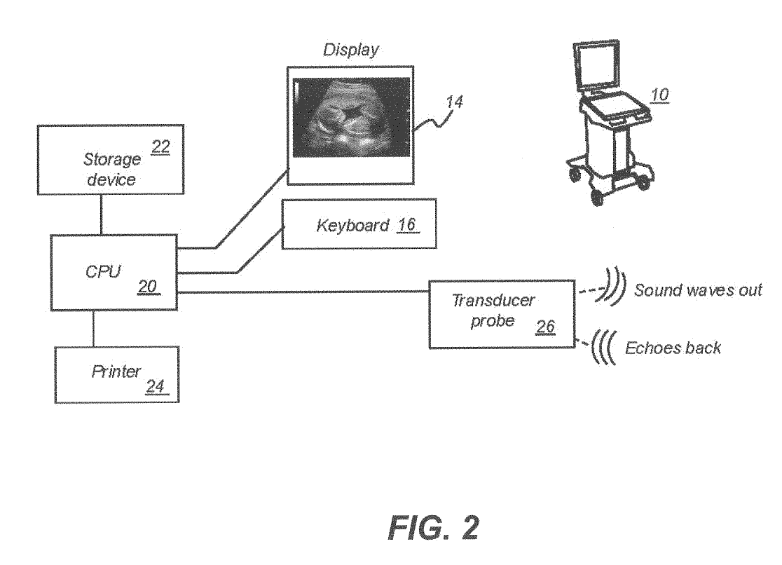

FIGS. 1A-1B and FIGS. 2-3 show exemplary portable ultrasound systems 10 that use a cart/base/support, cart 12, a display/monitor 14, one or more input interface devices 16 (such as keyboard or mouse), and a generator or beamformer 18 that is energizable to generate an ultrasound signal. The display/monitor 14 can also be a touchscreen to function as an input device. As illustrated, the ultrasound system 10 can be a mobile or portable system designed to be wheeled from one location to another. As FIG. 2 shows, the ultrasound system 10 has a central processing unit CPU 20, a control logic processor that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics from the echoed sound.

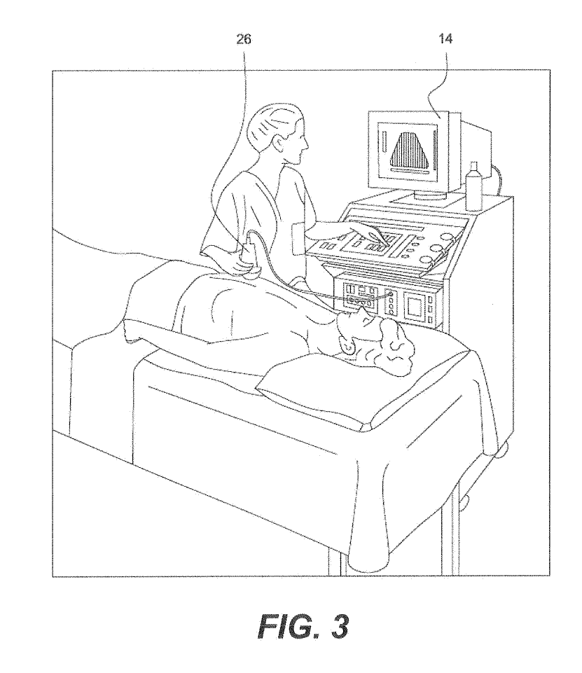

FIG. 3 shows an example of an ultrasound system 10 in use with an image provided on display/monitor 14.

Different types of images, with different appearance, can be formed using sonographic apparatus. The familiar monochrome B-mode image displays the acoustic impedance of a two-dimensional cross-section of tissue. Other types of image can use color or other types of highlighting to display specialized information such as blood flow, motion of tissue over time, the location of blood, the presence of specific molecules, tissue stiffness, or the anatomy of a three-dimensional region.

Accordingly, the ultrasound systems of FIGS. 1A-3 are typically configured to operate within at least two different ultrasound modes. As such, the system provides means to switch between the at least two different ultrasound modes. Such a multi-mode configuration, along with techniques for switching between modes, are known to those skilled in ultrasound technology.

The ultrasound system, shown by way of example in FIGS. 1A and 1B, can include an image processing system, a user interface, and a display. The image processing system includes a memory and a processor. Additional, different, or fewer components may be provided in the system or image processing system. In one embodiment, the system is a medical diagnostic ultrasound imaging system. The memory is a RAM, ROM, hard drive, removable media, compact disc, DVD, floppy disc, tape, cache memory, buffer, capacitor, combinations thereof or any other now known or later developed analog or digital device for storing information. The memory is operable to store data identifying a selected point for identifying a region of interest. The memory is operable to store data identifying one or a plurality of region of interest. Information from the user interface indicating a position on an image on the display is used to determine a spatial relationship of a user selected point to a scanned region or image position. The selected point is an individual or single point in one embodiment that may be a point selected within a line, area or volume. Additional or different information may be also stored within the memory. The processor is general processor, application-specific integrated circuit, digital signal processor, controller, field programmable gate array, digital device, analog device, transistors, combinations thereof, or other now known or later developed devices for receiving analog or digital data and outputting altered or calculated data. The user input is a track ball, mouse, joy stick, touch pad, buttons, slider, knobs, position sensor, combinations thereof or other now known or later developed input devices. The user input is operable to receive a selected point from a user. For example, the user positions a cursor on an image displayed on the display. The user then selects a position of the cursor as indicating a point for a region of interest. The display is a CRT, LCD, plasma screen, projector, combinations thereof or other now known or later developed devices for displaying an image, a region of interest, region of interest information and/or user input information.

Modes of ultrasound used in medical imaging include the following:
A-mode: A-mode (amplitude mode) is the simplest type of ultrasound. A single transducer scans a line through the body with the echoes plotted on screen as a function of depth. Therapeutic ultrasound aimed at a specific tumor or calculus is also A-mode, to allow for pinpoint accurate focus of the destructive wave energy.
B-mode or 2D mode: In B-mode (brightness mode) ultrasound, a linear array of transducers simultaneously scans a plane through the body that can be viewed as a two-dimensional image on screen. Sometimes referred to as 2D mode, this mode is generally the starting point for exam types that use other modes.
C-mode: A C-mode image is formed in a plane normal to a B-mode image. A gate that selects data from a specific depth from an A-mode line is used; then the transducer is moved in the 2D plane to sample the entire region at this fixed depth. When the transducer traverses the area in a spiral, an area of 100 cm² can be scanned in around 10 seconds.
M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.
Doppler mode: This mode makes use of the Doppler effect in measuring and visualizing blood flow.
Color Doppler: Velocity information is presented as a color-coded overlay on top of a B-mode image. This mode is sometimes referred to as Color Flow or color mode.
Continuous Doppler: Doppler information is sampled along a line through the body, and all velocities detected at each point in time are presented (on a time line).
Pulsed wave (PW) Doppler: Doppler information is sampled from only a small sample volume (defined in 2D image), and presented on a timeline.
Duplex: a common name for the simultaneous presentation of 2D and (usually) PW Doppler information. (Using modern ultrasound machines, color Doppler is almost always also used; hence the alternative name Triplex.).
Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.
Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

While conducting an ultrasound exam, the sonographer may often switch between multiple ultrasound modes. For example, the sonographer first operates in a B-mode in order to coarsely locate the ROI. The sonographer then transitions to a Doppler mode before moving back to the B-mode. For some particular examinations, there are pre-set (or pre-determined or pre-defined) steps/modes that the sonographer must follow. That is, the ordered sequence of modes used in a particular exam type can be predefined for the operator.

For carotid artery imaging, for example, the exam typically follows a progression of modes such as: (i) B-mode for initial positioning and establishing reference coordinates of the sample volume; (ii) Color Flow mode for improved visualization of blood vessels; and (iii) Pulse wave Doppler mode for highlighting blood flow within the sample volume.

For heart imaging, the exam progression can use B-mode or M-mode imaging for auto-positioning of the cursor, followed by Color Flow or pulse wave Doppler modes.

The Applicant has noted that in combination modes (such as Color Flow and Doppler), the sonographer preferably optimizes the settings for each of the modes individually. Also, based on the physical orientation of the anatomy on the displayed image, some of the settings are optimized on a per patient basis. This per patient optimization does not lend itself to global customization.

When viewing an ultrasound image on the display, the particular area of the displayed image that is of interest to the sonographer or other practitioner is referred to as the Region of Interest (ROI) or ROI extent. As the sonographer conducts the examination and switches between modes, the size and position, as well as the apparent shape of the ROI may change. This can require that the operator readjust settings in order to more accurately show features of anatomy in the ROI.

The region of interest (ROI) can be defined in any of a number of ways. In conventional practice, the ROI is defined by multiple points or vertices that define a shape, such as defining a rectangle or other parallelogram by its four corners, for example. Alternately, the ROI can be defined by a point and a distance, such as a center point and a radius or function of the distance from the point to a single boundary. The distance may be, for example, any of a radius, circumference, diagonal, length or width, diameter or other characteristic of a shape. The region of interest can alternately be defined by a point and two distances, such as a distance to each of two boundaries. In another arrangement, the region of interest can be a pre-defined shape positioned around a point, such as a square, rectangle, oval or combination thereof.

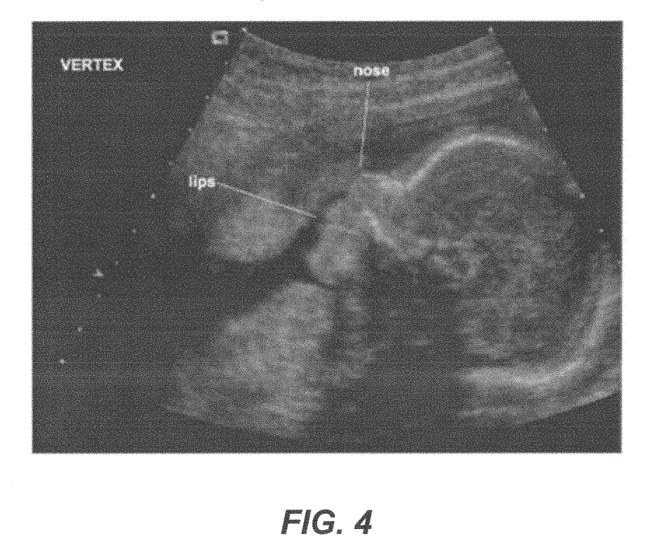

The sonography workflow typically begins with acquisition of a grayscale mode image acquisition and display (such as the B-mode image illustrated in FIG. 4) in order to survey the anatomy. Depending on the exam type, the operator then switches to a different imaging mode such as Color Doppler mode (sometimes referred to as Color Flow mode or Color mode) to evaluate a sub-region of the originally viewed grayscale image in order to obtain additional clinical information and further characteristics of the anatomy or tissue within a particular ROI. The ROI in a polychromatic or color imaging mode can be indicated by a rectangular, parallelogram, trapezoidal or another regularly shaped outline. In a typical ultrasound system, the spatial extent of the color ROI is a partial subset of the larger B-mode image; some portions of the B-mode image may not be displayed in the subsequent color mode. This is because the computational processing demands for polychromatic presentation are significantly higher than those for grayscale B-mode processing and rendering; this is among the tradeoffs commonly established in conventional practice.

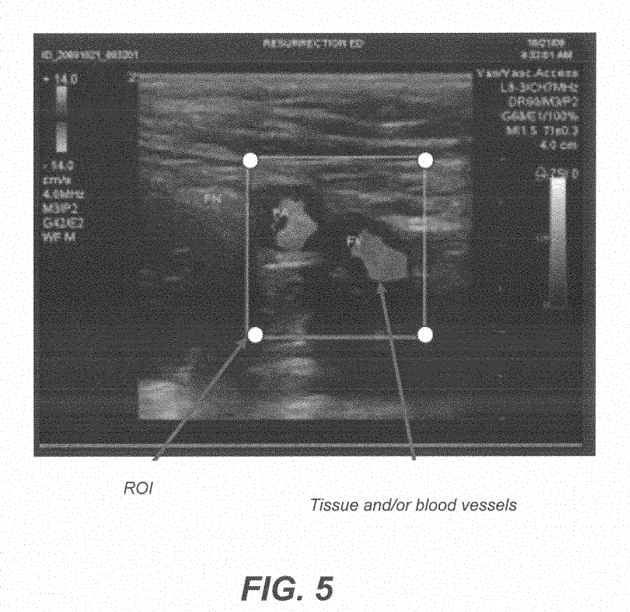

By way of example, FIG. 4 shows B-mode ultrasound image, displayed as a grayscale image. FIG. 5 shows an image with the same ROI having color highlighting, obtained in Color Flow mode.

A preferred embodiment can be described as a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The methods described above may be described with reference to a flowchart. Describing the methods by reference to a flowchart enables one skilled in the art to develop such programs, firmware, or hardware, including such instructions to carry out the methods on suitable computers, executing the instructions from computer-readable media. Similarly, the methods performed by the service computer programs, firmware, or hardware are also composed of computer-executable instructions.

### Portable Ultrasound Apparatus

Portable Ultrasound systems are known. The following references are incorporated herein by reference:
U.S. Pat. No. 7,534,211 entitled “Modular Apparatus for Diagnostic Ultrasound” to Hwang et al. (Ultrasound cart with docking station);
U.S. Pat. No. 9,180,898 entitled “Cart for Portable Ultrasonic Diagnostic Device and Ultrasonic Diagnostic Unit” to Ninomiya et al. (Ultrasound cart with flat surface);
US 2008/0161688 entitled “Portable Ultrasonic Diagnostic Imaging System with Docking Station” (Tablet connected to cart);
WO 2006/111874 entitled “Portable Ultrasonic Diagnostic Imaging System with Docking Station” to Poland et al;
WO 2016/001865 entitled “Portable Ultrasound Interface for Ultrasound Workstations” to Maraghoosh et al. (Tablet connected to cart);
WO 2008/068710 entitled “Dockable Ultrasound Systems and Method Thereof” to Brock-Fisher (Mobile Display); and
CN 102930170 entitled “Novel Interactive Medical Ultrasound Display and Input System” (Video Glasses).

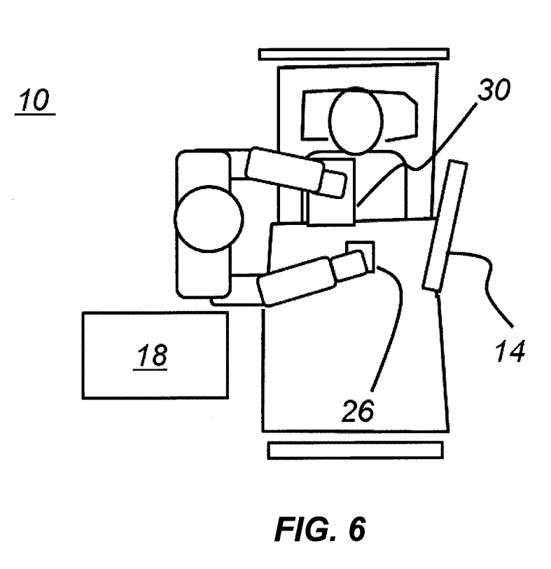

Applicants now refer to FIG. 6 describing an arrangement for a user interface (UI) and display for an ultrasound system, including a compact/portable ultrasound system, according to an embodiment of the present disclosure.

In one arrangement of an ultrasound system, there are separate units for Control Panel 30 and Display Monitor 14 wherein: (i) an External Control Panel 30 can be placed in an ergonomic location, and (ii) an External Display 14 can be placed ergonomically, for example, on the other side of the patient. The Ultrasound Engine (Computer) or generator, beamformer 18, can be a separate module or can be integrated with the control panel or monitor.

In a typical exam layout, the ultrasound technician (a) has right hand on transducer probe 26 on patient, (b) is turned 90+ degrees to left to look at monitor, that is, looking away from patient, and (c) has left hand on ultrasound control panel. For the patient, the monitor is difficult to see. Applicants have recognized that, for a more desirable exam layout, the ultrasound technician would have both arms aimed forward and would be facing the display monitor 14 and patient at same time; during the exam, the patient should be able to readily see the monitor 14. The ultrasound system 10 illustrated in FIG. 6 provides a desired exam layout.

### Haptics

An embodiment of the present disclosure can use haptic effects in order to configure the control panel, allowing controls to be readily located by the operator, using the sense of touch. The desired tactile stimulus can be accomplished using any of a number of effects, such as etched glass. Capacitive touch can be provided on a surface, whether metal, plastic or glass. Various control elements can be raised, formed, or etched on these surfaces. Haptic feedback allows the interface to dynamically provide feedback to the touching finger at encountering a raised edge or a control button. Haptics can allow increase or decrease in surface friction or the like, using high-frequency sound waves or electrical pulses. Microfluidic inputs can also be used, for example, as is shown at the web site immersion.com, under “haptics”.

There are haptic solutions using sound waves, electrical pulses or microfluidics. These technologies allow the “feel” or “texture” of the surface of the display to be changed dynamically. The system can alternate between: displaying an image with no imperfections in the surface of the display; showing some buttons along the bottom that feel like buttons; showing a full, standard QWERTY keyboard with a hundred buttons taking up most of the screen. In all cases, the feel of the buttons can change based on what is being displayed.

Using haptic effects, on-screen controls can be configurable or customizable based on exam type, user, mode or the like.

Menus or fly outs can include additional information displayed on the main monitor. For example, clicking a button on the control panel generates a list of options, shown on the monitor that displays the image. The user can scroll through and select an option. This aspect can be useful when the control panel display does not have the required space, or where there is no corresponding display; scroll wheel or track ball to quickly move through a list of options. Informational or control display can appear on the control panel or main display.

The display need not be flat like conventional control panels. The display, for example, could be fist-shaped, shaped like a computer mouse, or held manually in the manner of a WII remote or video game controller. The shape can be configured to facilitate finding buttons by touch.

On-screen controls can be any of the following: buttons, sliders, or joysticks. Controls can be twisted, scroll wheels, track ball, and track pad.

From an ergonomic standpoint, the control panel can be spaced apart from the display and need not be elevated, allowing its placement on the patient's bed, for example.

The system can be wired or wireless or both and can use the well-known Bluetooth or WIFI interface. The system can be battery operated, such as using a rechargeable battery. A USB cable can provide both battery recharge and provide a wired data path when plugged in. A power status indicator can be provided. A wireless connectivity indicator can be provided, along with controls or mechanisms to enable Bluetooth pairing.

The system can be easy to clean, having an IP68 rating, for example, and can be rubberized. Capacitive metal, stainless steel, plastic or glass can be used, including glass with flexible flat panel display technology or curved surface. The transducer can be wireless.

The system can be docked to a device that also holds the display monitor, including a dock that recharges the control panel, for example.

The system can be wired when connected to the control panel. A dock can hold or store transducers. The dock can be on a rolling system that itself has a larger battery, plugs into the wall, and has its own charging system. The control panel can be docked to the same device that holds the display that is normally on the other side of the patient.

### Motion Sensing Control Panel

A motion sensing control panel can be employed, held in the user's hand for operation. Alternatively, motion sensing could use a glove or similar device that is worn instead of held. The motion sensing device can use a wireless connection.

Motion sensing can use an inclinometer to sense change in angle, motion, or acceleration in any or all of 6 axes, similar to a WII remote device. Motion sensing can employ buttons, joysticks, track ball, sliders or use different controls under different fingers. The device can contain a small display, light emitting diodes (LEDs) or other light elements to indicate status. Another secondary display could: (i) provide feedback on what the operator is doing with the hand-held control panel or (ii) show current status, function of each control on the hand held in current state. This could provide feedback (for confirmation or failure) to user actions, such as audible feedback or vibration feedback.

### External Monitor

An external monitor can be separate from the control panel so that it can be placed in a more ergonomic location, for example, on the opposite side of bed from the operator. The form factor can be optimized, such as using a tablet style display with a kick-stand mounting to seat on a table-top or other surface. The mount can be coupled to an intravenous (IV) pole or device or can mount to a cart or other device.

The ultrasound engine or computer could be also mounted to an IV pole or to a cart. For pole mounting, a low position would provide a low center of gravity, helping to prevent the patient from knocking the device over. If the monitor can be removed from the pole, then it is more likely that the ultrasound engine could also be mounted to the back of the monitor, forming a single unit that can be carried around. The monitor can pivot left/right up/down (separate from or with ultrasound engine enclosure), such as to address glare issues. Angle adjustment can be based on sitting height of operator vs. height of monitor. The mount can pivot the display using buttons/sliders on the control panel, so that operator need not move around the patient bed to adjust the mount.

The monitor can move up or down on a pole for better viewing position, such as varied with operator height. It may include a manual release, reengagement brake, or clamp at a suitable minimum height so that the display does not drop to the floor. The monitor can rotate to portrait or landscape orientation, such as with its view orientation varied according to the exam type. In a preferred arrangement, the monitor recognizes its physical orientation automatically and adjusts content of the user interface to match the orientation.

Contents of the display can be user-, exam-, or mode-configurable. The operator can suspend transducers on pole, cart, or storage compartment in the display or ultrasound engine. A single pane of glass would provide easy cleaning.

The display can extend away from pole or cart. For example, the pole or cart can be placed at the base of the patient bed, instead of along the opposite side of the bed, and can then telescope over the bed.

The arrangement can allow the operator to always use the control panel with right hand instead of needing to operate ambidextrously. Room layout and possible locations of hardware need not force the operator to sit on a specific side of the bed. It can be easier to position the monitor away from patient if desired.

The monitor can be attached to the operator, an arrangement that could be useful with other technologies such as fluoroscopy for tube placement. For example, it can be attached to the operator's wrist or hung around the neck. On a portable system, additional monitor display space can be obtained by having a second monitor that folds down, hinged like a laptop, or that slides out from a side of the primary display.

There are alternatives to the traditional control panel and/or monitor. These include (i) wearable touchscreen monitor, for example on wrist or hung around neck, (ii) glove, such as finger motions instead of buttons, make fist, open palm, and motion sensing for entire hand, moving the hand to control ultrasound, (iii) virtual/augmented reality, (iv) voice, and (v) handwriting recognition.

Features of the control panel can include: patient entry or selection; probe selection; protocol selection; preset selection; calculations; report generation; operating frequency; PRF; image size; gain; dynamic range; mode selection, freeze, store, image review, and image send-to-destination instructions.

The display can use a projector to project the obtained image on a wall or other surface. Alternatively, it can be a projector integrated into the ultrasound engine or computer; control panel; or standalone unit tethered to computer (wired or wireless).

The device preferably has a handle for easy carrying. It preferably has a control panel that docks or snaps into the ultrasound engine, similar a tablet having a removable and attachable keyboard to for a laptop. It preferably has a portable printer or DVD burner incorporated into an IV pole or cart or standalone devices that can be attached to computer or can be wireless. The device preferably has hangers or storage for one or more transducers.

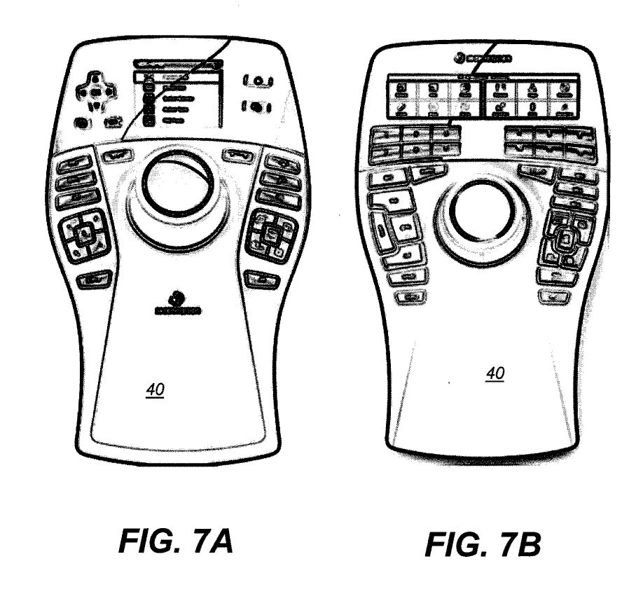

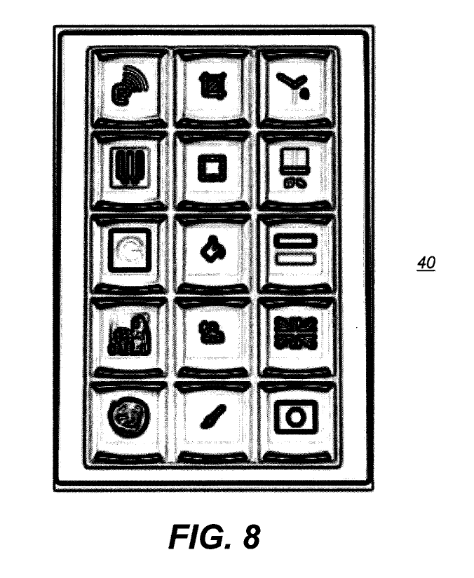

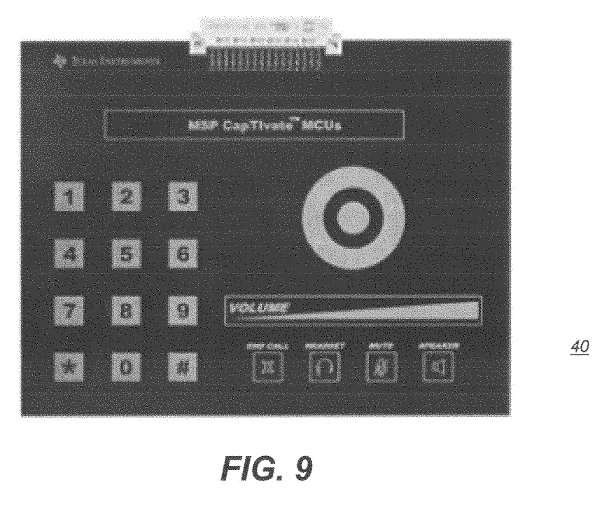

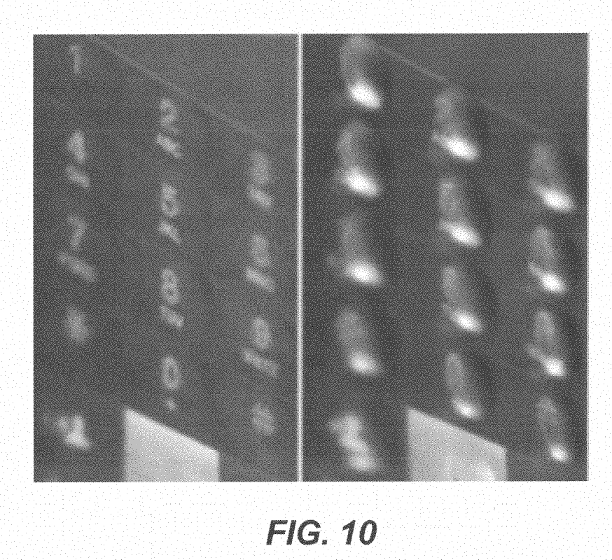

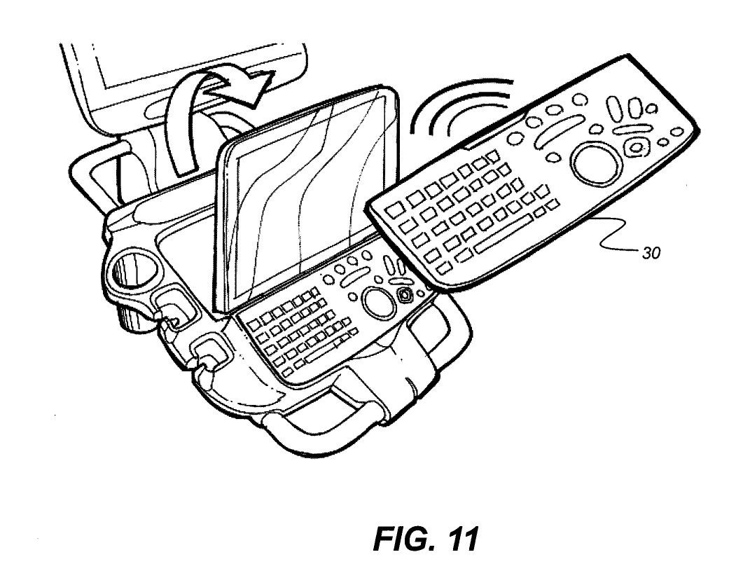

Preferably, the carrying case can hold a control panel; display plus ultrasound engine plus computer, all in one, and/or transducers. Optionally, the control panel docks into the ultrasound engine. There can be a wearable item worn by the operator to hold the system or part of the system, such as utility belt; hung around the neck; holders for transducers; display in front of the operator; this can be battery operated. Examples of controllers 40 which might be suitable are shown in FIGS. 7A, 7B, 8, and 9. FIG. 10 shows haptic raised buttons, for example.

### Removable Control Panel

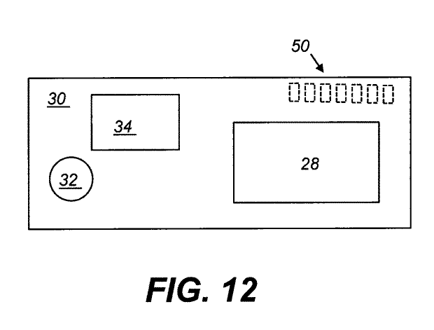

FIG. 11 is a perspective view that shows a removable control panel. The control panel 30 can be in wireless signal communication with the controlling processor 20 of the ultrasound system.

Applicants' ultrasound system can employ a control panel 30 (when it is not a basic tablet) that is preferably detachable from other equipment of the ultrasound system 10, as shown in FIG. 11. The display monitor 14 can, for example, be mounted on another support, such an intravenous (IV) pole/cart with the rest of the ultrasound cart, engine, or PC across on the other side of the patient's bed.

The operating control panel 30 can be an external device, such as a USB connected device such as a mouse or trackball with buttons and other types of controls. In various arrangements, a connecting wire runs to the ultrasound engine or beamformer 18, computer or CPU 20 as the control logic processor; alternately, the control panel contains the ultrasound engine and computer. The control panel 30 can be placed anywhere on any surface. It is preferably placed where it is ergonomically favorable for the ultrasound technician. With this arrangement, there is no requirement for the sonographer to be ambidextrous in order to be able to work from different sides of the patient.

The operating control panel 30 can be flat or have a raised back. A kick stand or similar type of hinged device can be provided for support. Alternately, a bean bag-style back can be provided to add weight, to adapt to a non-flat surface (bed), or to hold more easily in place, or more easily adjust angle. The material used for support can be plastic, vinyl or similar material that can be easily cleaned.

With the operating control panel, buttons on the control panel can be conventional control buttons and sliders. For example, backlit buttons can be provided for use in low light; these can be rubberized for easy cleaning. There can be a small built in display on the control panel. There can be configurable buttons within the display, such as using a small liquid-crystal device (LCD) screen. For operation, the user can touch the screen directly or use an adjacent control button. Alternatively, the whole surface of the control panel can be a display or touch screen. The surface can have tactile qualities that allow identification of buttons by touch.

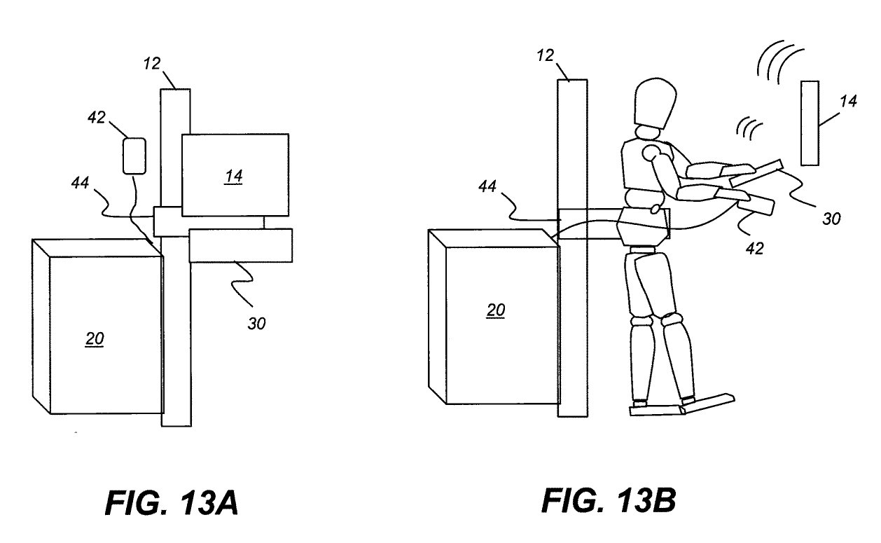

According to an embodiment, piezoelectric transducers are used to generate haptic stimuli that can help the user to identify locations of buttons or controls. Able to provide suitable local vibration along any position of the control panel, these actuators can allow the operator to find, by touch, the position of keyboard and other controls at suitable locations on the control panel 30, as shown in FIG. 12. Piezoelectric actuators 50 can be selectively or globally distributed beneath the glass or other smooth surface of the control panel, providing keys and controls at operator-selected locations, including providing the capability for the operator to adjust the locations or angles of controls for improved accessibility as the operator moves to different positions about the patient. Using haptic stimulation can help to guide the operator to using the configurable control panel 30 more effectively, guided by feel as well as visual indicators. According to an optional embodiment, the control panel 30 can be curved or have other than a flat surface for improved ergonomics.

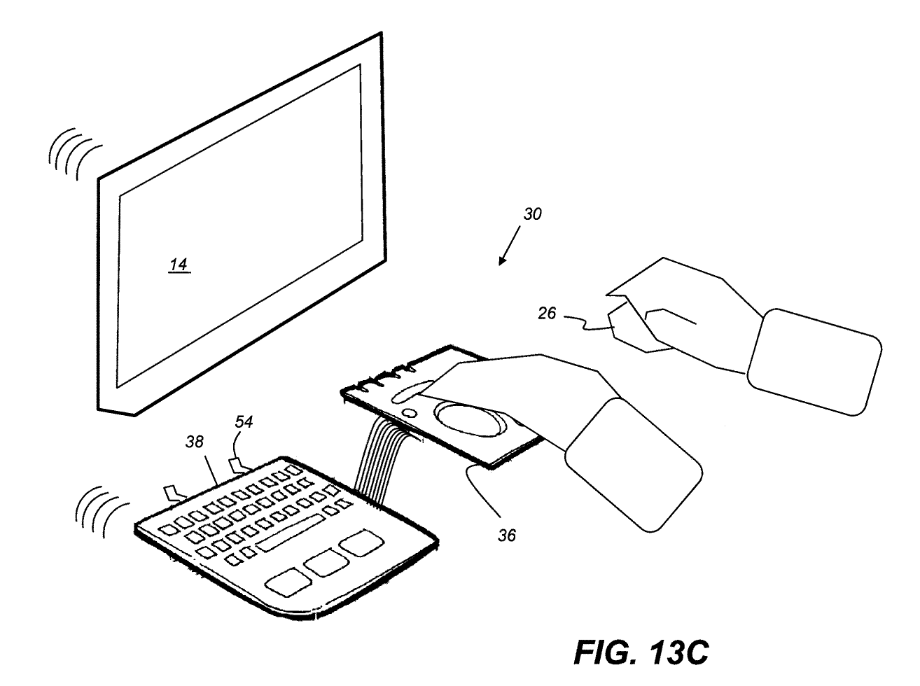

FIG. 13A shows ultrasound system 10 having a cart 12, a display monitor 14, a transducer 42, CPU 20, control panel 30, and a docking station 44 for seating and providing recharging capability. FIGS. 13B and 13C show display monitor 14 and control panel 30 in a wireless configuration, removed from docking station 44 and in use for acquiring ultrasound images. Wireless communication with the control logic processor, CPU 20, enables control functions for acquisition and display of ultrasound data. As is shown in FIG. 13C, control panel 30 can be split into two portions, a contoured or etched portion 36 having a surface that is etched or otherwise treated or provided with haptic stimuli for locating at least first and second fixed-position controls according to touch and a touchpad portion 38 for example, for ease of use by the operator. This arrangement helps to reduce the size of the control panel features that are more difficult to fabricate, isolating these features to the etched portion 36, for example. Features that can use standard touchpad logic are then provided on the associated touchpad portion 38. Another advantage of a segmented control panel 30 relates to how sonographer controls are actually used. By separating controls that are generally used during the active scan from those used for pre-scan configuration and setup, this segmented and wireless arrangement allows considerable flexibility for the operator. Improved accessibility to controls and the capability to place controls where they are most convenient can help to accommodate left-handed as well as right-handed sonographers and to tailor the system configuration for different exams and patient characteristics. Etched portion 36 is typically featured to enhance touch detectability of gain and other controls that are often adjusted during an exam. Featuring can provide etched or raised control elements, for example.

With portions 36 and 38 separated, one or the other portions can be used to provide hinged support for display monitor 14. FIG. 13C shows a hinge 54 provided along an edge of touchpad portion 38. Hinge 54 can be used to support display monitor 14 at an operator-selectable angle.

The removable wireless portions 36 and 38 and, optionally, display monitor 14 can use on-board battery power for operation and for maintaining the wireless communication link. Batteries can be rechargeable or non-rechargeable. For recharging, connection ports on the ultrasound system can provide the needed power when control panel 30 sections are docked. The same source can be used for battery power to both portions. One or both of the portions can have the circuitry for providing wireless communication with the control logic processor.

### Wireless Recharging

Alternately, motion charging or non-contact recharging can be provided.

With respect to wireless recharging, reference is hereby made to the following references, incorporated herein by reference in their entirety:

U.S. Pat. No. 8,115,448 entitled “Systems and Methods for Wireless Power” to Johns;

U.S. Pat. No. 6,127,799 entitled “Method and Apparatus for Wireless Powering and Recharging” to Krishnan;

U.S. Pat. No. 7,383,064 entitled “Recharging Method and Associated Apparatus” to Mickle et al; and

US 2015/0214765 entitled “Focus Control for Wireless Power Transfer” to Perry.

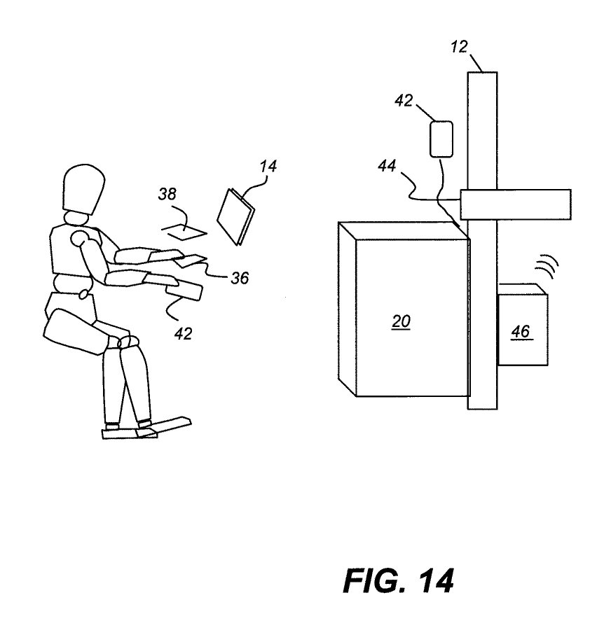

As shown schematically in FIG. 14, an omni-directional power transmission device 46 located on the mobile ultrasound cart transmits energy via radio frequency signals (i.e. 2.4 GHz, 5.8 GHz) to removable wireless portions 36 and 38, to optionally wireless display monitor 14, and to other specifically configured electronic devices within a specified range (typically up to about 30 feet with commercially available devices). Power transmission is relatively independent of orientation and location on, in, or near the cart (also independent of whether the device being charged is stationary or moving) to be wirelessly recharged. The associated wireless power receiver in each corresponding rechargeable electronic device then converts this received RF signal into battery power. Recharging can be very efficient, even charging at the same rate as if the device were plugged into a wall outlet or other electrical power connection.

A wireless charging system may be enabled for “on demand” charging of each wireless device, meaning that the RF transmitter will only transmit an RF signal to the wireless device when the wireless device receiver transmits a recharge request (for example, when the wireless device detects a power drop below 50% of its battery capacity). Standard size batteries are also available with these built-in receivers so that devices using standard batteries can also be recharged using this method. This can be advantageous for the mobile ultrasound cart, since other devices may be carried on the cart, such as a flashlight, handheld pulse oximeter, or other battery powered medical or non-medical devices. Because this method of charging is enabled up to a 30 foot radius (and the charging distance itself can be configurable in some cases), the control panel portions 36, 38 have more charge time available during use, not simply when idle. That is, these devices can actually be charged while in operation. For instance, a control panel battery can be charging while the patient ultrasound exam is taking place.

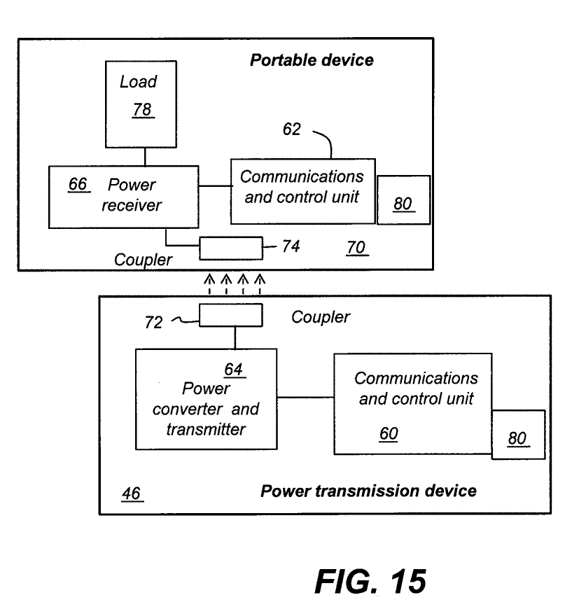

The schematic diagram of FIG. 15 shows some basic components for a wireless charging system that uses inductive charging or a similar wireless charging mechanism. Power transmission device 46, which can be provided on the ultrasound cart as described previously, has a communications and control unit 60 and a power converter 64. Communications and control unit 60 coordinates and controls generation and delivery of the oscillating electromagnetic signals that are wirelessly transmitted for providing power. Power converter 64 provides an electromagnetic signal to a coupler 72, such as an inductive coupler, for transmission. High frequency transmission can be used to provide the power signal.

On a portable device 70, such as a wireless control panel 30 or wireless portions 36 and/or 38 as described previously or other compatible device, a corresponding wireless coupler 74 accepts the transmitted electromagnetic signal, which is converted to electrical energy for use by a load 78 such as for battery charging or directly used. A communications and control unit 62 coordinates and controls power monitoring and delivery over the wireless coupling. The respective communications and control units 60 and 62 can be dedicated processors that handle power and recharging functions or can be processing logic components that control other functions for their respective devices. Communications and control units 60 and 62 can include a transponder 80 for communication between power transmission and portable devices.

Frequencies used for charging can be appropriately specified in order to reduce the likelihood of interference to neighboring equipment. In general, the frequencies used are in the bandwidth range of frequencies typically used for mobile phone communication (for example, 2.4 GHz), widely used among patients and staff in the hospital setting.

As described previously, the wireless power transmission device 46 of the present disclosure can be energized to deliver a power-providing RF signal to an associated ultrasound device that is configured to receive a preselected frequency. When not within range of a compatible device needing charge, communications and control unit 60 can enter a standby state to reduce energy consumption.

The control panel surface can be flat or can have one or more curved regions.

### Alarm for Loss of Wireless Link

According to an alternate embodiment of the present disclosure, wireless control panel 30, separable portions 36 and 38, and wireless display monitor 14 can be provided with an alarm or illumination behavior, such as blinking, that indicate loss of wireless communication with the control logic processor, CPU 20. This can help to prevent inadvertent separation of these wireless components from ultrasound system 10, such as at the completion of an exam.

According to an alternate embodiment of the present disclosure, a second display can be provided, such as for patient viewing.

Transducer 42 button features can include commands to effect a number of operations, including the following: Freeze; Store; Acquire Cine; 3D activate; Biopsy; CW Doppler; Color; Elasto; PW Doppler; Smart Flow; Zoom.

Control panel 30 features can include: Patient entry or selection; Probe selection; Protocol Selection; Preset Selection; Calculations; Report generation; Operating Frequency; PRF; Image size; Gain; Dynamic Range; Mode (choosing among available modes: B-Mode, color, Pulse Wave, Motion Mode, 3D, and the like); Freeze; Store; Image Review; Image Send to destination.

### Augmented Reality Configurations

According to an alternate embodiment, augmented reality devices, such as head-mounted displays (HMDs) can be used by the sonographer or by the patient, for viewing acquired images. For the patient, for example, this can include features that allow controlled visibility of acquired image content.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### Clean Feature

Portable Ultrasound systems are known, and the following references are incorporated herein by reference: U.S. Pat. No. 7,534,211 (ultrasound cart with docking station), U.S. Pat. No. 9,180,898 (ultrasound cart with flat surface), US20080161688 (tablet connected to cart), WO2006111874A2, WO2016001865A1 (tablet connected to cart), WO2008068710A1 (mobile display), CN102930170A (video glasses). All of these references are incorporated herein in their entirety by reference.

An ultrasound system may include one or speakers. For example, referring again to FIGS. 1A and 1B, an ultrasound system can include one or more speakers as well as a control panel with a plurality of elements. In some configurations of speakers, a speaker grill is very difficult to clean, particularly a configuration which includes a plurality of small holes. In ultrasound systems having a tablet style arrangement, the single screen on the front of the tablet style is both a touch screen control panel used for image display. Tablets usually have tiny speakers on the front or the rear with a single small hole.

It is recognized that regular cleaning on medical devices is required to prevent the spread of infectious disease. A typical ultrasound control panel contains elements which are touched by a Sonographer (such as buttons, sliders, track ball) of which would therefore preferably be readily cleanable. Applicants recognize the desirability to clean all aspects of an ultrasound system.

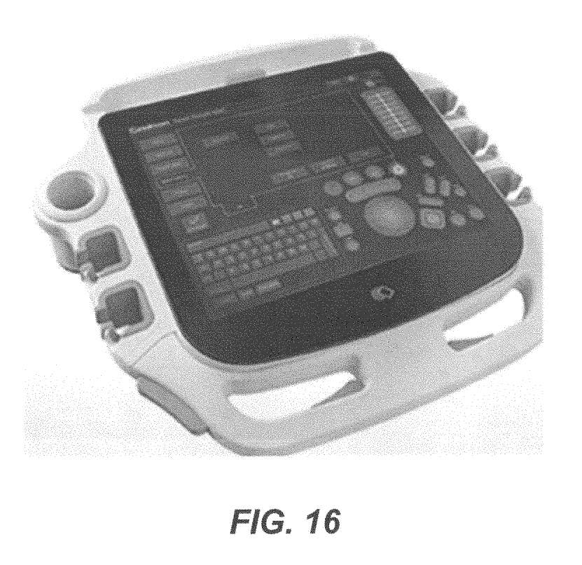

An ultrasound system having an etched glass control panel promotes cleanability. Such an ultrasound system is illustrated in FIG. 16. With etched glass, the control panel surface is all glass, but the glass is etched so that the Sonographer can operate the control panel by feel/touch/tactile instead of by vision. This allows the operator to focus their attention on the image monitor and the patient.

Applicants have developed a portable ultrasound system using an etched glass control panel. It employs a dockable/removable etched glass control panel. In one arrangement, there is a portable (wired or wireless) control panel (not primarily for image display) using an etched glass control panel. Etched buttons can be configured to have different features, but recognize that the location of a button/element cannot be changed.

A control panel with Haptic Feedback allows for easy cleaning and customization. Haptic systems dynamically provide feedback to the touching finger the feeling of a raised edge, a button, more/less friction, using sound waves, electrical pulses, microfluidics. It provides the feeling of a button or of the control even though the surface of the control panel is flat. Ex: Immersion.com Haptics.

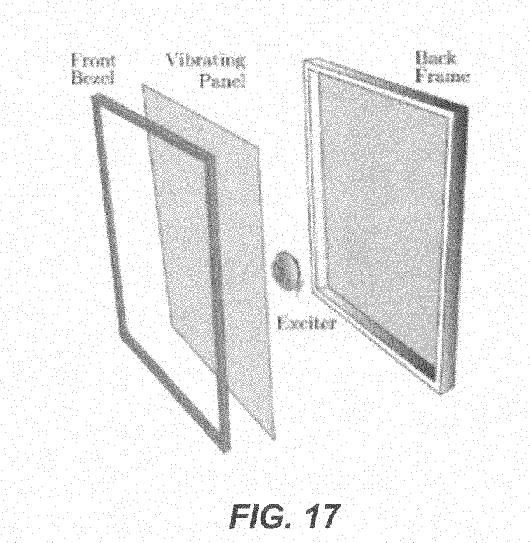

Referring FIG. 17, another way to promote cleaning ability is to replace standard speakers and speaker grills with a distributed mode loudspeaker (DML)/Flat Panel Speaker. Flat panel speakers are a piece of rigid plastic or other material that vibrates. No speaker grill is required, so no holes are needed to let the sound pass through. The flat panel speaker concept applies to various medical device with a speaker where cleaning is an issue.

### Customized Login

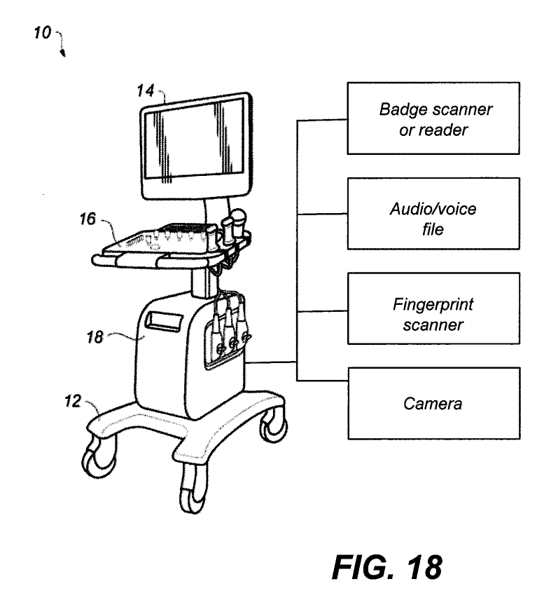

FIG. 18 shows an ultrasound system including one or more of the following: a camera; finger print reader/scanner; an audio or voice reader/scanner/detector; a badge/identification reader/scanner.

Applicants have recognized the desirability for customizing the login and operating preferences of an ultrasound system. The customization of the login and/or preferences can be accomplished using any one of the elements: a camera; finger print reader/scanner; an audio or voice reader/scanner/detector; a badge/identification reader/scanner.

The customization could allow the operator to conduct/accomplish one or more of the following: (i) to identify themselves at the system (via user name/password, proximity badge, biometrics, and the like); (ii) to login to the system after being successfully identified, such as gain access to PC operating system and/or medical application; (iii) to configure the system based on operator preferences; (iv) to identify scanning preferences, including customized values for acquisition parameters; customized layout of ultrasound control panel; (v) to define Reading preferences, including Controls for the display of image information; (vi) control/define image processing preferences, including controls for the processing of images before the images are displayed; (vii) define information, including: colors, brightness; screen layout; and workflow layout (patient input or patient selection; patient/exam lists; image review; send image to destination).

Preferences which can be pre-set by means of the customized login include presence, location, method of selection or input of the following controls (not limited to): Probe selection; Protocol Selection; Preset Selection; Calculations; Report generation Operating Frequency; PRF; Image size; Gain; Dynamic Range; Mode Selection (choosing among available modes: B-Mode, Color, Pulse Wave, Motion Mode, 3D); Freeze; and Store.

Customized login also allows for the Configuration of Control Panel, which includes: placing/moving controls; grouping multiple controls in drop down/fly out menus, popup windows; changing the style of the controls—buttons vs. rocker switch/sliders vs. knobs.

The user has ability to change the preferences for themselves. Administrator users have ability to change the preferences or set the default preferences for groups/categories of users or all users.

It is noted that a second operator (such as a Radiologist, Administrator, or the like) could come to look at an image, swipe their badge, and if so, the settings change for that new user, including Control Panel Layout, Image Processing changes, and the like.

It is noted that, with the arrangement as described, the system includes unique configurations per user/per mode/per exam. In some arrangements, the Same user has different control panel layout for brightness vs. Color Doppler mode. In other arrangements, the same user has different control panel layout for cardiology vs. Gynecology

It is noted that the system can be shipped with factory configurations for each mode and exam type.

What is claimed is:

1. An ultrasound apparatus comprising:
a beamformer energizable to generate an ultrasound signal;
a control logic processor;
a transducer probe configured to direct and detect ultrasound signals;
a control panel in wireless signal communication with the control logic processor; and
a display monitor in signal communication with the control logic processor.

2. The ultrasound apparatus of claim 1 wherein the control panel comprises two separable portions:
a first portion having a surface treated for locating at least first and second fixed-position controls according to touch; and
a second portion having a touch screen that displays variable controls according to selection from the at least first and second fixed-position controls on the first portion.

3. The ultrasound apparatus of claim 2 wherein one of the first and second portions provides the wireless signal communication with the control logic processor and wherein the first and second portions are in signal communication with each other.

4. The ultrasound apparatus of claim 2 wherein the treated surface provides a haptic stimulus using one or more piezoelectric actuators.

5. The ultrasound apparatus of claim 2 wherein the treated surface is etched.

6. The ultrasound apparatus of claim 1 wherein the control panel portions are formed from glass.

7. The ultrasound apparatus of claim 1 wherein one or both of the control panel portions are dockable for recharging.

8. The ultrasound apparatus of claim 1 wherein the display monitor surface or control panel is curved.

9. The ultrasound apparatus of claim 1 further comprising a head-mounted display for the sonographer or patient.

10. The ultrasound apparatus of claim 1 further comprising a docking station for recharging of the control panel or display monitor.

11. The ultrasound apparatus of claim 1 further comprising a viewer display for patient viewing.

12. The ultrasound apparatus of claim 2 wherein one or both portions are hinged to support the display monitor when undocked.

13. The ultrasound apparatus of claim 1 further comprising a recharging power transmitter that performs wireless recharging.

14. The ultrasound apparatus of claim 13 wherein at least a portion of the control panel is configured for wireless recharging.

15. An ultrasound apparatus comprising:
a beamformer;
a control logic processor;
a transducer probe configured to direct and detect ultrasound signals;
a control panel having first and second portions, the first and second portions being separable from each other for operation of the apparatus, the first or second or both portions being in wireless signal communication with the control logic processor;
a recharging power transmitter adapted to perform wireless recharging; and
a display monitor in signal communication with the control logic processor.

16. The ultrasound apparatus of claim 15 wherein one of the first and second portions are rechargeable using wireless transmission from the recharging power transmitter.

17. An ultrasound apparatus comprising:
a beamformer;
a control logic processor;
a transducer probe configured to direct and detect ultrasound signals;
a control panel in wireless signal communication with the control logic processor;
a recharging power transmitter adapted to perform wireless recharging; and
a display monitor in signal communication with the control logic processor.

18. The ultrasound apparatus of claim 17 wherein the control panel further provides motion sensing for entry of operator instructions.

19. The ultrasound apparatus of claim 17 wherein the control panel has a hinged support.
